(19) United States
(12) Patent Application Publication
(10) Pub. No.: US 2010/0016265 A1
(43) Pub. Date: Jan. 21, 2010

(54) Anti-inflammatory composition and method for preparation

(76) Inventor: Qaiser Yusuf, League City, TX (US)

Correspondence Address: Michael L. Parks;Parks & Associates, PLLC, Suite 101, 3131 W. Alabama, Houston, TX, 77098, US

(21) Appl. No.: 12/218,771

(22) Filed: Jul. 16, 2008

(51) Int. Cl. A61K 31/573; A61K 31/27; A61K 31/164; A61P 29/00

(52) U.S. Cl. 514/171; 514/538; 514/626

(57) ABSTRACT

A pharmaceutical anti-inflammatory preparation and method for preparation and treatment of arthritis and related inflammatory conditions used for mild to severe pain for relatively long acting results. This anti-inflammatory preparation comprises anti-inflammatory corticord steroid in aqueous-like suspension, a muscle relaxant in oil-like composition, and amide anesthetic compound in aqueous-like composition and Vitamin B-12 in aqueous-like composition. It is prepared by mixing the anti-inflammatory preparation at a rate and for a time sufficient to create a suspended emulsion. The anti-inflammatory preparation is administered while in suspended emulsion by intramuscular patient injection.

### FIELD OF THE INVENTION

This invention relates generally to an anti-inflammatory preparation of pharmaceutically effective agents for the treatment of arthritis and related inflammatory conditions by intramuscular injection composed of anti-inflammatory corticordsteroid in an aqueous-like suspension; a muscle relaxant in an oil-like composition and amide anesthetic compounds in an aqueous-like composition. A general method of preparation of these pharmaceutically effective agents is mixing the anti-inflammatory preparation with sufficient agitation/rate of agitation and for a time sufficient to create a suspended emulsion, which is intramuscularly injected into a patient while the preparation is in suspended emulsion.

### BACKGROUND OF THE INVENTION

It is well known in the prior art that corticordsteroids, both natural and synthetic, may be used in the treatment of inflammatory, painful conditions such as injuries, osteoarthritis, rheumatoid arthritis, fibromyalgia and similar acute conditions such as acute spinal and joint injuries. Generally these corticordsteroids were used in relatively high doses, which gave immediate results to the patient, however, those patients with chronic or progressive conditions of pain quickly exceeded the dosage limit of corticordsteroids. These dosage limits restrict their continued use on patients and physicians ran out of ammunition or options for continued treatment once the very large doses have been administered. The use of corticordsteroids has also been accompanied by the varied use in different combinations and compositions of muscle relaxants, anti-inflammatories, and anesthetic agents; however, the administration of these additional compounds has been haphazard in the terms of the amount of dosages that are used for the treatment of acute or chronic pain conditions/inflammatory conditions and they have been generally used to reduce the side effects of the corticordsteroid. Clearly, the prior art was not directed toward a synergistic effect which would allow the reduction in the dosage levels of corticordsteroids used for patients in the treatment of inflammatory painful conditions which would allow more effective treatment at low dosage levels and provide the ability to provide long-term treatment and still not exceed the dosage limits of corticordsteroids.

The prior art has attempted to deal with solving some of the side effects of steroids by adding calcium, based on the amount of a particular steroid used either in solution or suspension to offset bone loss from the use of the corticordsteroids which can lead to osteoporosis. Also in conjunction with the calcium addition the prior art used Vitamins B-12, C and/or D for the purpose of facilitating the calcium absorption and utilization. Even further, some prior art has used anesthetics such as procaine, lidocaine, etc. with corticordsteroids and calcium to reduce the site-specific pain of injection. Yet other of these combinations have been administered by injecting intra-articularly, and intramuscularly and orally ingesting these combinations, but these prior art combinations had short duration of effectiveness with patients. These other combinations required frequency of administration from daily to weekly to be effective.

Other approaches have been the use of loading the active ingredient corticordsteroids into liposomes, such as polyethyleneglycol (“PEG”), by dissolving the corticosteroid in aqueous phase sufficient to stay in and reach encapsulation in as high a concentration as possible in the relatively bio-inactive liposomes. The purpose of PEG-liposomes in this prior art was to deliver the water-soluble corticosteroid at the desired site of inflammation. The process for this encapsulation required a complex chemical process and then the liposomal suspension was transferred to an extruder and treated under pressure, using nitrogen gas, six times through two filters having a pore size of 200 nm and 100 nm respectively and 100 nm and 50 nm and 50 m and 50 nm respectively. This complex process achieved a particle size with a range of 40 nm to 200 nm and remained stable in a nitrogen atmosphere at 4° C. for approximately two months. This process and product gave improved results over using straight corticordsteroid, but failed to deal with the other side effects and dosage level problems associated with steroid use in patients.

Yet other prior art used corticordsteroids in combination with different nonsteroidal anti-inflammatory agents such as aspirin and skeletal muscle relaxants to provide systemic treatment for the relief of arthritis pain. Some of the other non-steroidal agents used were phenylbutazone, calcium carbonate, and Vitamin D. The agents, calcium carbonate and Vitamin D, were used to offset possible negative effects of osteoporosis, which are common with the use of corticosteroids. The muscle relaxant used was methocarbamol at relatively high dosages in a range of 500 mg to 1000 mg. In this prior art, there was no special preparation of the mixture and in its broadest sense it was suggested that unit dosages could for example be delivered as tablets, powders, pills, capsules, and the like and still be within the scope of the invention. Clearly there was no concept of using a special preparation technique to create the preparation mixture prior to its administration to achieve effectiveness at reduced corticosteroid and muscle relaxant levels and/or dosages.

The prior art in many cases used common dosages of corticosteroids and simply added compounds to control the side effects of the corticosteroids or to add nonsteroidal anti-inflammatory agents to the composition to join with the corticosteroids for an anti-inflammatory treatment of chronic pain. The prior art did not look for interaction between the components, except in the case of calcium carbonate and Vitamin D to prevent osteoporosis caused by continued use of corticosteroids, and simply added compounds together with each doing its independent anti-inflammatory function without regard to the other components. The prior art did not teach that various combinations of anti-inflammatory agents used together could reduce the dosage of each of these anti-inflammatory agents and get a superior result in patients using lower dosages because of synergistic effects. Clearly, the prior art did not contemplate the use of lower dosages and combining the mixture with a simple process which makes the lower dosages in combination with each other more effective in systemic treatment of chronic pain.

Because of the lack of effectiveness of the prior art anti-inflammatory compounds, patients often supplemented their treatment with narcotics/opiates and oral muscle relaxants and anxiolytics which have toxic side effects and reduce a patient's quality of life even further. Also these prior art anti-inflammatory compounds required some physical therapy/rehabilitation regimen to be effective for patients and this interfered with a patient's work.

Many of the prior art compounds were expensive drugs and required a pharmaceutical preparation before they could be administered and some even required special storage conditions to keep their effectiveness and they could not be readily reconstituted thereafter. Also these prior art anti-inflammatory medications could be difficult to administer and required multiple administrations to be effective for any sustained period of time. Because of the multiple injections the cost of administering the drugs was substantially expensive and further was made difficult to administer. It required a skilled person to administer the drugs because it required injection as an intra-articularly or tendon sheath injection.

### BRIEF SUMMARY OF THE INVENTION

This invention provides an anti-inflammatory preparation for intramuscular injection and method for preparation prior to injection which has long-lasting effects for the treatment of arthritis and related inflammatory pain conditions. The preparation uses pharmaceutically approved agents in a novel combination and at lower doses than normally used and yet achieves inflammatory pain management with long-lasting effects even at the lower doses. This improved pain management result is achieved at such low doses that in most cases, the dosages do not exceed the approved limits for patient over a sustained period of time and can be continued without significant side effects. Also because of the low dosages of each of the compounds, side effects are significantly reduced, especially for the corticordsteroid.

The anti-inflammatory preparation of this invention comprises an anti-inflammatory corticordsteroid in an aqueous-like composition; a muscle relaxant in an oil-like composition; and an amide anesthetic compound or compounds in an aqueous-like composition mixed together at a sufficient rate of agitation and for sufficient time to create a suspended emulsion. The suspended emulsion is intramuscularly injected into the patient while in the suspended emulsion state. In some compositions of this anti-inflammatory preparation, Vitamin B-12 is added before mixing the composition into an emulsion so that the Vitamin B-12 is also emulsified prior to the injection of the emulsion into a patient for treatment.

### DESCRIPTION OF THE PREFERRED EMBODIMENTS

In the present invention, it has been discovered that a synergistic effect is achieved in combining lower dosages of corticordsteroid, which include glucocorticordsteroids, such as Methylprednisolone, in aqueous-like composition; muscle relaxants, such as Methocarbamol, in an oil-like composition; a short acting amide anesthetic, such as Lidocaine, in an aqueous-like composition; a long-acting amide anesthetic, such as Marcaine, in an aqueous-like composition; and transforming the mixture of all the above compositions into a suspended emulsion by mixing the combined compositions at a sufficient agitation rate and for a sufficient time to create a suspended emulsion. In some compositions of this anti-inflammatory preparation, Vitamin B-12 is added before mixing the composition into an emulsion so that the Vitamin B-12 is also emulsified prior to the injection of the emulsion into a patient for treatment.

Further, it has been discovered that the synergistic effects are further enhanced by the treatment of patients with an intramuscular injection of the suspended emulsion. The intramuscular injection provides a systemic treatment effect for the treatment of arthritis and related inflammatory conditions.

The synergistic effects of the injection of this emulsion composition are for the purposes of reducing the pain of arthritis-related inflammatory conditions and these compositions other than corticordsteroid, which include glucocorticordsteroids such as Methylprednisolone, are not to offset the side effects of the corticordsteroid, but to act with corticordsteroid at the inflamed tissues to allow the lower dosage of the corticordsteroid to be more effective at the treatment of the inflammatory conditions and to provide longer lasting results to the patient with fewer side effects.

It has been found that in the broadest sense, the synergistic effects of the inventive preparation of one embodiment of this invention were obtained for patients with mild to most severe pain in the dosage range of 10 mg to 120 mg of Methylprednisolone in an aqueous-like suspension; 25 mg to 150 mg of Methocarbamol in an oil-like composition; 2.0 mg to 25 mg of Lidocaine in an aqueous-like solution; 2.0 mg to 25 mg of Marcaine in an aqueous like solution and up to 750 mcg of Vitamin B-12 in an aqueous-like solution when this combination was mixed at a sufficient rate of agitation and for a time sufficient to create a suspended emulsion of these compounds. Further, it has been found that for the patients to receive the full benefit of these compounds that the patient must be injected while these compounds are in the suspended emulsion state.

It has further been found that a treatment regimen can be developed to fit the patient's conditions, which allows treatment for mild, moderate, severe, and most severe pain and still achieve a long-acting result for each of these classes of patients.

The treatment regimen for patients with mild pain used a range of 10 mg to 30 mg of Methylprednisolone in an aqueous-like suspension; 25 mg to 60 mg of Methocarbamol in an oil-like composition; 2.0 mg to 15 mg of Lidocaine in an aqueous-like solution; 2.0 mg to 20 mg of Marcaine in an aqueous-like solution; and up to 500 mcg of Vitamin B-12 in an aqueous-like solution.

Patients with moderate pain were effectively treated with a range of 25 mg to 60 mg of Methylprednisolone in an aqueous-like suspension; 25 mg to 60 mg of Methocarbamol in an oil-like composition; 2.0 mg to 15 mg of Lidocaine in an aqueous-like solution; 2.0 mg to 20 mg of Marcaine in an aqueous-like solution; and up to 500 mcg of Vitamin B-12 in an aqueous-like solution.

While patients with severe pain were treated with a regimen in a range of 30 mg to 80 mg of Methylprednisolone in an aqueous-like suspension; 40 mg to 100 mg of Methocarbamol in an oil-like composition; 5.0 mg to 20 mg of Lidocaine in an aqueous-like solution; 5.0 mg to 20 mg of Marcaine in an aqueous-like solution; and up to 750 mcg of Vitamin B-12 in an aqueous-like solution.

Finally, patients with the most severe pain were treated with a regimen in the range of 60 mg to 120 mg of Methylprednisolone in an aqueous-like suspension; 50 mg to 150 mg of Methocarbamol in an oil-like composition; 5.0 mg to 25 mg of Lidocaine in an aqueous-like solution; 5.0 mg to 25 mg of Marcaine in an aqueous-like solution; and up to 750 mcg of Vitamin B-12 in an aqueous-like solution.

In the process of testing these ranges on patients and in determining a specific dosage for each patient, they were divided into categories of pain and designated DR-1 mild pain, DR-2 moderate pain, DR-3 severe pain, and DR-4 most severe pain. More than 1,200 patients were treated in the process of determining a more specific range for each patient in these categories. As those skilled in the medical arts will appreciate, the dosage ranges may be adjusted for each particular patient depending on the patient's response after the first series of administered injections, however, at least one preferred clinical starting dosage was determined for each DR-1, DR-2, DR-3, and DR-4 as follows:

| | |
|---|---|
| DR-1 | 20 mg Methylprednisolone |
| | 50 mg Methocarbamol |
| | 5 mg Lidocaine |
| | 5 mg Marcaine |
| | 250 mcg Vitamin B-12 |
| DR-2 | 40 mg Methylprednisolone |
| | 50 mg Methocarbamol |
| | 5 mg Lidocaine |
| | 5 mg Marcaine |
| | 250 mcg Vitamin B-12 |
| DR-3 | 60 mg Methylprednisolone |
| | 75 mg Methocarbamol |
| | 10 mg Lidocaine |
| | 10 mg Marcaine |
| | 500 mcg Vitamin B-12 |
| DR-4 | 80 mg Methylprednisolone |
| | 100 mg Methocarbamol |
| | 10 mg Lidocaine |
| | 10 mg Marcaine |
| | 500 mcg Vitamin B-12 |

These compounds were mixed at a mixing rate of 10 Hz for five minutes or until the mixture was homogeneously mixed into an emulsion and no longer exhibited separate states, as for example in water-like and oil-like separate states. The mixing process occurred at nominal room temperature. Properly mixed, these dosages may stay in emulsion for up to 10 days, but if prior to intramuscular injection the physician finds the emulsion has begun to separate into its water-like and oil-like components, it may be re-emulsified by re-agitation for a time sufficient to fully emulsify the composition prior to it being administered to the patient.

At least one source of Methylprednisolone, which has been used to make up some of these compositions, is DEPO-MEDROL, which is the Trademark of Pharmacia Corp. and at least one source of Methocarbamol, which has been used to make up some of these compositions, is ROBAXIN, which is the Trademark of Baxter Healthcare Corporation. Also at least one source for Lidocaine and Marcaine, which have been used to make up some of these compositions, is Hospira, Inc. While at least one source for vitamin B12 (Cyanocobalamin), which has been used to make up some of these compositions, is from American Regents Inc.

In clinical trials with the dosages levels of DR-1, DR-2, DR-3 and DR-4 above set out were used on 100 patients in each DR group selected by their pain levels to correspond to the appropriate DR group. The following were run:

DR-1 dosage for mild recurrent cases of arthritis/inflammatory conditions and/or acute traumatic conditions such as acute strains or contusion to the joints or spine was administered to a total of 50 patients with the exclusion of Vitamin B-12, and another 50 patients with the inclusion of Vitamin B-12. The average effect from one intramuscular injection gave lasting pain relief of between two to four weeks duration, depending on the individual patient. The onset of the effectiveness occurred within 15 minutes to 30 minutes. The side effects are minimal and generally patients may exhibit increased energy levels and slight heartburn. Their appetites may be slightly increased for a period of less than 24 hours. The vast majority of patients preferred the Vitamin B-12 included in their dosage as they described improved energy levels and less fatigue and tiredness than the group without the Vitamin B-12 added. Since the dose of Methylprednisolone is a very low dose and duration effect is quite long, one skill in the art would understand that you can comfortably administer up to eight to ten of these injections in a 12 month period and still only be at a total combined dosage of Methylprednisolone of less than 200 mg.

DR-2 dosage was confined to patients with moderate inflammatory conditions/osteoarthritis/rheumatoid arthritis/fibromyalgia and was administered to a total of 50 patients with the exclusion of Vitamin B-12, and another 50 patients with the inclusion of Vitamin B-12. The average effect from one intramuscular injection gave lasting pain relief of between three to six weeks duration, depending on the individual patient. The onset of the effectiveness occurred within 15 minutes to 30 minutes. Since the dose of Methylprednisolone is still a low dose and duration effect was quite long, one skilled in the art would understand you can comfortably administer these injections almost monthly over a 12-month period and still only be at a total combined dosage of Methylprednisolone of less than 400 mg. The predominant side effects of this dosage were mild heartburn/acid reflux symptoms which were generally temporary lasting about 48 to 72 hours after administration of the DR-2 dosage. This side effect was easily alleviated by the use of antacids or acid reducing agents for a period of one week to ten days after administration of the injection. The vast majority of patients preferred the Vitamin B-12 included in their dosage as they described improved energy levels and less fatigue and tiredness than the group without the Vitamin B-12 added.

DR-3 dosage was confined to patients with acute exacerbations of chronic inflammatory conditions and/or severe contusions/strain/sprains to the extremities, joints or spine and was administered to a total of 50 patients with the exclusion of Vitamin B-12, and another 50 patients with the inclusion of Vitamin B-12. The average effect from one intramuscular injection gave lasting pain relief of between four to six weeks duration, and 90% of the patients and six to eight weeks in 10% of patients. The onset of the effectiveness occurred within 15 minutes to 30 minutes. Since the dose of Methylprednisolone is a low dose and duration effect was quite long, one skilled in the art would further understand that you can comfortably administer these injections almost monthly over a 12-month period and still only be at a total combined dosage of Methylprednisolone of less than 400 mg to 600 mg. The predominant side effects of this dosage were mild heartburn/acid reflux symptoms which were generally temporary lasting about 48 to 72 hours after the administration of the DR-3 dosage. This side effect was easily alleviated by the use of antacids or acid reducing agents for a period of one week to ten days after administration of the injection. The other side effect in about 20% of the patients was mild weight gain of less than five pounds. The weight gain however, is mainly attributed to the lack of activity in patients due to their severe arthritic conditions. Those patients that remained active on a daily basis and exercised regularly did not show a significant weight gain. The vast majority of patients preferred the Vitamin B-12 included in their dosage as they described improved energy levels and less fatigue and tiredness than the group without the Vitamin B-12 added.

DR-4 dosage was confined to patients who had received cortisone injections on numerous occasions in the past at high dosages and who also suffered very severe inflammatory conditions which were causing significant disability and significant compromise of daily activities. This included patients who were using canes, walkers, or were wheelchair-bound as a result of their chronic inflammatory/arthritic conditions and included patients who had multiple surgeries to spine, extremities, or joints as a result of herniated discs, arthritis of the spine, or joint or ligament or tendon injuries. It was administered to a total of 50 patients with the exclusion of Vitamin B-12, and another 50 patients with the inclusion of Vitamin B-12. The onset of the effectiveness occurred within 15 minutes to 30 minutes. The dose of Methylprednisolone administered was reserved for very severe cases of inflammatory conditions and the effect was a 30 to 40% perceived pain reduction in the patients. One skilled in the art would understand that you can comfortably administer these injections in short bursts for severe conditions up to three to five times over a 6-month period for a total dosage of 300 mg to 400 mg and still only be at a total combined dosage of Methylprednisolone of less than 800 mg over 12 months. The predominant side effects were heartburn/acid reflux symptoms after the administration of the DR-4 dosage. This side effect was treated by putting these patients on H-2 blockers to prevent gastritis /acid reflux disease during the occurrence of these side effects of the DR-4 dosage. The other side effect in about 20% of the patients was weight gain of five to ten pounds over a one year period. The weight gain however is mainly attributed to the lack of activity in patients due to their severely impaired physical conditions. Also as many of these patients, because of their other medical conditions and treatments, are on steroids in other forms for their other conditions, it compounded the total steroid dose to a much higher level, but still stayed under a total dosage of 800 mg per year. However these DR4 patients were able to successfully reduce their amounts of narcotic and opiate medications along with the reduction of nonsteroidal anti-inflammatories thereby reducing renal toxicity and gastric toxicity. The vast majority of patients preferred the Vitamin B-12 included in their dosage as they described improved energy levels and less fatigue and tiredness than the group without the Vitamin B-12 added.

An additional benefit from all the different combinations of DR-1, DR-2, DR-3, and DR-4 use was a reduction in the amount of narcotics/opiates used, generally ranging from between 30 to 60%. There was also a reduction in amount of oral muscle relaxants used from anywhere between 50 to 70% and a reduction in the amount of anxiolytics used by 50 to 70%. These reductions improved the quality of life, activities of daily living, and function without toxic side effects from oral pain medications, muscle relaxants and anxiolytics. Also these combinations were effective for patients even when patients did not comply with the two to three times a week of physical therapy recommended either due to disability or psychosocial problems, or they just continued to go to work rather than take off for physical therapy.

All these patients receiving DR-1, DR-2, DR-3, and DR-4 were objectively monitored with x-rays, CAT scans, MRI scans, EMG/NCV to see the extent of their disease progression and they were regularly monitored on a monthly basis and the data was collected to determine the long-term efficacy of these medications in reducing their long term progression of the disease process. From the data, it was determined that the disease process of the patients was slowed in many cases, but even when the disease process was not slowed there was improvement in mobility and activities of daily living and function in the patients. While the exact mechanism for the synergistic effect of lower dosages of each pharmaceutically effective agent when combined as taught in this invention is not fully understood, they never the less give longer-lasting results than when used as individual agents at higher dosages. There are at least some theories about how these combinations of medications and their method of preparation and treatment may work.

At least one theory suggests that inflamed tissue has less ability to uptake corticordsteroids and that less inflamed tissue can uptake corticordsteroids more readily, thereby, reducing the concentration of corticordsteroids necessary to be present for tissue uptake to achieve the same medicinal effect. The effect of the emulsion created by the method of preparation of these compounds and their injection while in an emulsified state, provides even distribution of the dosage of all the compounds and holds them in an emulsion for some time after their injection into the patient. Therefore the combination of effective agents as prepared by the method taught in this invention is believed to provide immediate pain reduction with the anesthetic compounds, both with the short-acting and long-acting, and keeps the tissue from feeling inflamed and then it allows the muscle relaxant to further relax the tissue into its most relaxed state for the highest possible uptake of the corticordsteroids even though present at low dosages. It is about effective uptake of corticordsteroids without having systemically high doses of corticordsteroids.

At least another theory is that the method of preparation of these compounds into the emulsion causes corticordsteroids in an aqueous-like composition to adsorb to the surface of the muscle relaxant in an oil-like composition and serve as a carrier for some portion of the corticordsteroids and the combination targets abnormally inflamed tissue, sparing normal tissue. The portions of the muscle relaxant not serving as a carrier causes inflamed tissue to relax and subsequently allows uptake of the muscle relaxant and the corticordsteroids adsorbed to it into the tissue for further tissue relaxation and the slow release of the adsorbed corticordsteroids over time.

While the above are some theories of the reactive mechanism of this invention may not be totally accurate, there may be many more and different reactive mechanisms which are actually at work with the combination of these anti-inflammatory agents taught by this invention for the treatment of arthritis and related inflammatory conditions.

While the preferred compositions of this anti-inflammatory invention for the treatment of inflammatory pain conditions and the method of their preparation and the treatment for patients have been described, it will be appreciated that other composition embodiments and methods and treatment procedures may be used without departing from the spirit of the inventions and that they may be altered in a manner within the skill of the art without departing from the scope of the inventions as defined in the claims herein claimed.

That claimed is:

1. An anti-inflammatory preparation of pharmaceutically effective agents for the treatment of arthritis and related inflammatory conditions including mild to most severe pain for relatively long acting results comprising of:
a. anti-inflammatory corticordsteroid in an aqueous like suspension;
b. a muscle relaxant in an oil like composition; and
c. an amide anesthetic compound in an aqueous like composition.

2. An anti-inflammatory preparation of claim 1, wherein said amide anesthetic compound further comprises:
a. a short acting amide anesthetic in an aqueous like composition; and
b. a long acting amide anesthetic in an aqueous like composition.

3. An anti-inflammatory preparation of claim 2, wherein said anti-inflammatory corticordsteroid further comprises:
a. an anti-inflammatory glucocorticordsteroid in an aqueous like suspension.

4. An anti-inflammatory preparation of pharmaceutically effective agents of claim 3 wherein said anti-inflammatory glucocorticordsteroid in an aqueous like suspension is selected from the group consisting of:
a. Methylprednisolone, Prednisolone, Prednisone, and Triamcinolone.

5. An anti-inflammatory preparation of pharmaceutically effective agents of claim 4, wherein said muscle relaxant in an oil like composition further comprises:
a. Methocarbamol, Dantrolene, Diazepam, Metaxalone, Carisoprodol, Baclofen, Norflex, and Flexeril.

6. An anti-inflammatory preparation of pharmaceutically effective agents of claim 5, wherein said short acting amide anesthetic compound in an aqueous like composition and long acting amide anesthetic compound in an aqueous like composition respectively comprises:
a. Lidocaine; and
b. Marcaine.

7. An anti-inflammatory preparation of pharmaceutically effective agents of claim 6, for mild to most severe pain wherein said preparation further comprises:
a. 10 mg to 120 mg of Methylprednisolone in an aqueous like suspension;
b. 25 mg to 150 mg of Methocarbamol in an oil like composition;
c. 2.0 mg to 25 mg of Lidocaine in an aqueous like composition; and
d. 2.0 mg to 25 mg of Marcaine, in an aqueous like composition for the treatment of arthritis and related inflammatory conditions from mild, to most severe pain for relatively long acting results.

8. An anti-inflammatory preparation of pharmaceutically effective agents of claim 7, further comprising:
a. Vitamin B-12 in an aqueous like composition.

9. An anti-inflammatory preparation of pharmaceutically effective agents of claim 8, wherein said Vitamin B-12 in an aqueous like composition further comprising:
a. Up to 750 mcg of Vitamin B12 in an aqueous like composition.

10. A method for preparation of said anti-inflammatory preparation of claim 9, comprising:
a. mixing said anti-inflammatory preparation at a sufficient rate and for a time sufficient to create a suspended emulsion.

11. A method for treatment of patients with said anti-inflammatory preparation of claim 10 comprising of:
a. intramuscular injecting of said anti-inflammatory preparation into a patient while said suspended emulsion of said anti-inflammatory preparation is in said suspended emulsion.

12. A method of preparation of said anti-inflammatory preparation of claim 10, wherein said mixing rate sufficient and time sufficient to create a suspended emulsion, further comprises:
a. an oscillation rate of at least 5 Hz; and
b. a time of at least 1 minute for creating said suspended emulsion of said anti-inflammatory preparation in suspended emulsion for a time period of up to 12 hours for intramuscular injecting into a patient while remaining in said suspended emulsion.

13. An anti-inflammatory preparation of pharmaceutically effective agents for the treatment of arthritis and related inflammatory conditions of claim 7, wherein said treatment for mild pain for relatively long acting results comprises:
a. 10 mg to 30 mg of Methylprednisolone in an aqueous like suspension;
b. 25 mg to 60 mg of Methocarbamol in an oil like composition;
c. 2.0 mg to 15 mg of Lidocaine in an aqueous like composition; and
d. 2.0 mg to 20 mg of Marcaine, in an aqueous like composition.

14. The anti-inflammatory preparation of claim 13, further comprising:
a. Up to 500 mcg of Vitamin B12 in an aqueous like composition.

15. An anti-inflammatory preparation of pharmaceutically effective agents for the treatment of arthritis and related inflammatory conditions claim 7, wherein said treatment for moderate pain for relatively long acting results comprises of:
a. 25 mg to 60 mg of Methylprednisolone in an aqueous like suspension;
b. 25 mg to 60 mg of Methocarbamol in an oil like composition;
c. 2.0 mg to 15 mg of Lidocaine in an aqueous like composition; and
d. 2.0 mg to 20 mg of Marcaine, in an aqueous like composition.

16. The anti-inflammatory preparation of claim 15, further comprising:
a. Up to 500 mcg of Vitamin B12 in an aqueous like composition.

17. An anti-inflammatory preparation of pharmaceutically effective agents for the treatment of arthritis and related inflammatory conditions claim 7 wherein said treatment for severe pain for relatively long acting results comprises:
a. 30 mg to 80 mg of Methylprednisolone in an aqueous like suspension;
b. 40 mg to 100 mg of Methocarbamol in an oil like composition;
c. 5.0 mg to 20 mg of Lidocaine in an aqueous like composition; and
d. 5.0 mg to 20 mg of Marcaine, in an aqueous like composition.

18. The anti-inflammatory preparation of claim 17, further comprising:
a. Up to 750 mcg of Vitamin B12 in an aqueous like composition.

19. An anti-inflammatory preparation of pharmaceutically effective agents for the treatment of arthritis and related inflammatory conditions claim 7, wherein said treatment for most severe pain for relatively long acting results comprises:
a. 60 mg to 120 mg of Methylprednisolone in an aqueous like suspension;
b. 50 mg to 150 mg of Methocarbamol in an oil like composition;
c. 5.0 mg to 25 mg of Lidocaine in an aqueous like composition; and
d. 5.0 mg to 25 mg of Marcaine, in an aqueous like composition.

20. The anti-inflammatory preparation of claim 19, further comprising:
a. Up to 750 mcg of Vitamin B12 in an aqueous like composition.

21. A method for preparation of said anti-inflammatory preparation of claim 13, 15, 17, and 19, comprising:
a. mixing said anti-inflammatory preparation at a sufficient rate and for a time sufficient to create a suspended emulsion of said anti-inflammatory preparation.

22. A method for preparation of said anti-inflammatory preparation of claim 21, wherein said mixing rate sufficient and time sufficient to create a suspended emulsion, further comprising:
a. an oscillation rate of at least 5 Hz; and
b. a time of at least one minute for creating said suspended emulsion of said anti-inflammatory preparation in suspended emulsion for a time period of up to 12 hours for intramuscular injection into a patient while remaining in said suspended emulsion.

23. A method for treatment for patients with said anti-inflammatory preparation of claim 22, comprising of:
a. intramuscular injecting of said anti-inflammatory preparation into a patient while said suspended emulsion of said anti-inflammatory preparation is in said suspended emulsion.
